(19) United States
(12) Patent Application Publication
(10) Pub. No.: US 2007/0213378 A1
(43) Pub. Date: Sep. 13, 2007

(54) Compounds for modulating cell proliferation, compositions and methods related thereto

(75) Inventors: Helmut Thomas, Sherbrooke (CA); Leticia M. Toledo-Sherman, Venice, CA (US)

Correspondence Address: FISH & NEAVE IP GROUP;ROPES & GRAY LLP, ONE INTERNATIONAL PLACE, BOSTON, MA, 02110-2624, US

(73) Assignee: LymphoSign Inc., Markham (CA)

(21) Appl. No.: 11/716,398

(22) Filed: Mar. 9, 2007

Related U.S. Application Data
(60) Provisional application No. 60/781,128, filed on Mar. 10, 2006.

(51) Int. Cl. A61K 31/433; A61K 31/426; A61K 31/4245; A61K 31/422

(52) U.S. Cl. 514/362; 514/364; 514/365; 514/374; 514/341; 514/342

(57) ABSTRACT

Compounds which are useful in treating a variety of cell proliferative disorders such as cancer are disclosed.

### CROSS-REFERENCE TO RELATED APPLICATIONS

This application claims the benefit of U.S. Provisional Application No. 60/781,128, filed Mar. 10, 2006, the specification of which is hereby incorporated by reference in its entirety.

### BACKGROUND OF THE INVENTION

A wide range of growth factors coordinate cell proliferation and differentiation. Malignant cells arise as a result of a stepwise progression of events that include the unregulated expression of growth factors or components of their signaling pathways. Tyrosine phosphorylation events initiated by receptor, cytoplasmic and nuclear kinases and regulated by phosphatases are central to these processes. Mutation, hyper-activation, translocation and overexpression of protein tyrosine kinases are all associated with tumorigenesis. In addition to increasing proliferative rates and immortalizing cells, overexpression of tyrosine kinases can lead to morphological transformation and cause anchorage independence, contributing to the promotion of migratory ability and possibly the induction of metastases.

Certain compounds with structures based upon mimicry of ATP or phosphotyrosine have been shown to be effective kinase inhibitors. Those based upon phosphotyrosine have been demonstrated to be the more specific tyrosine kinase inhibitors. Because of their ability to inhibit tyrosine phosphorylation, these compounds may alter cell responses to growth factors or other processes driven by tyrosine kinase activity, including unregulated growth as the result of tyrosine kinase overexpression, mutation, or translocation. Inhibition of tyrosine kinases occupying a central role in proliferative signaling pathways, or in pathways regulating cell cytoskeletal structure, even temporary or incomplete inhibition, may be sufficient to switch a cancerous cell from a proliferative cycle into programmed cell death, or apoptosis. Death by apoptosis is most often observed upon effective treatment with tyrosine kinase inhibitors.

Selective inhibition of specific tyrosine kinases offers a method of targeting cancerous cell growth with a high degree of specificity and minimal toxicity to normally growing cells and tissues. Thus, specific inhibitors of tyrosine kinases have great potential as clinical anti-cancer treatments. A number of small molecules which act as tyrosine kinase inhibitors have been identified. For example, certain phenyl acrylonitrile compounds have been described as tyrosine kinase inhibitors, effective to inhibit cell proliferation (see for example, U.S. Pat. No. 5,891,917, U.S. Pat. No. 5,217,999, U.S. Pat. No. 5,773,476, U.S. Pat. No. 5,935,993, U.S. Pat. No. 5,656,655, U.S. Pat. No. 5,677,329 and U.S. Pat. No. 5,789,427).

Inhibition of tyrosine kinases offers one mechanism by which cell proliferation can be inhibited. One of skill in the art will appreciate that other mechanisms of inhibition may also be involved.

There is a need in the art to identify compounds that inhibit cell proliferation.

### SUMMARY OF THE INVENTION

A number of compounds have now been identified that inhibit abnormal cell proliferation, for example cancer cell growth. The compounds do not inhibit the growth of normal cells. Accordingly, the present invention relates to compounds of Formula I or a salt, solvate or hydrate thereof:

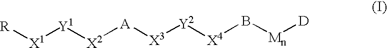

wherein, as valence and stability permit,

R is a substituted or unsubstituted alkyl, alkenyl, or alkynyl group or a substituted or unsubstituted carbocyclyl, heterocyclyl, aryl, or heteroaryl group;

A is a substituted or unsubstituted aryl or heteroaryl group;

B is a substituted or unsubstituted heteroaryl group;

D is a substituted or unsubstituted carbocyclyl, heterocyclyl, aryl, or heteroaryl group;

X¹, X², X³, and X⁴ each independently represent a direct bond, NR′, or O;

Y′ and Y², independently, each represent C═O, C═S, C═NR′, or SO₂, preferably C═O or SO₂;

M, independently for each occurrence, is a substituted or unsubstituted methylene group, NR′, O, or S, preferably selected so that no two heteroatoms occur in adjacent positions, more preferably with at least two carbon atoms between any nitrogen atom and another heteroatom;

R′, independently for each occurrence, is H or a substituted or unsubstituted lower alkyl group, preferably H or unsubstituted lower alkyl; and

n is an integer from 1 to 6,

or a pharmaceutically acceptable salt thereof.

In certain embodiments, R is a substituted or unsubstituted alkyl group, such as a substituted or unsubstituted lower alkyl group, for example, a methyl, ethyl, propyl or isopropyl group. In some embodiments, R is a substituted alkyl group, for example, an alkyl group substituted by one or more rings, such as carbocyclyl, aryl, heterocyclyl, or heteroaryl group. In further embodiments, R is a substituted or unsubstituted alkenyl or alkynyl group. For example, R may be an alkenyl group substituted by one or more groups such as carbocyclyl, aryl, heterocyclyl, or heteroaryl.

In some embodiments, A comprises a substituted or unsubstituted 6-membered ring. In certain embodiments, A is a substituted or unsubstituted aryl group, such as a substituted or unsubstituted benzene ring. In some of such embodiments, X² and X³ are disposed in a meta relationship on the benzene ring.

In certain embodiments A is unsubstituted. In alternate embodiments, A is substituted, for example, with one or more groups selected from halogen, nitro, cyano, hydroxyl, thiol, carboxyl, sulfate, substituted or unsubstituted amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, alkyl, alkenyl, alkynyl, acyl, acyloxy, acylamino, aryl, heteroaryl, carbocyclyl, heterocyclyl, aralkyl, hetaralkyl, carbocyclylalkyl, and heterocyclylalkyl.

In certain embodiments, B comprises a substituted or unsubstituted 5-membered ring. In some embodiments, B comprises 1-3 heteroatoms selected from O, S, and N. For example, B may be a substituted or unsubstituted thiophene, furan, pyrrole, pyrazole, imidazole, triazole, tetrazole, thiazole, isothiazole, thiadiazole, oxazole, isoxazole, or oxadiazole group, particularly a thiadiazole group. In some instances where B comprises a substituted or unsubstituted 5-membered ring, X⁴ and Mₙ are disposed in a 1,3 relationship on B.

In some embodiments, B is unsubstituted. In alternate embodiments, B is substituted with one or more groups selected from halogen, nitro, cyano, hydroxyl, thiol, carboxyl, sulfate, substituted or unsubstituted amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, alkyl, alkenyl, alkynyl, acyl, acyloxy, acylamino, aryl, heteroaryl, carbocyclyl, heterocyclyl, aralkyl, hetaralkyl, carbocyclylalkyl, and heterocyclylalkyl.

In certain embodiments, B represents 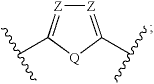
where Z, independently for each occurrence, is N or CR″, particularly N; Q is NR′, O, or S, particularly O or S; and R″, independently for each occurrence, is H or a substituted or unsubstituted lower alkyl group. In some embodiments, Z is N for both occurrences of B.

In certain embodiments, D is a substituted or unsubstituted aryl group, for example, a substituted or unsubstituted phenyl group.

In some embodiments, D is unsubstituted. In alternate embodiments, D is substituted with one or more groups selected from halogen, nitro, cyano, hydroxyl, thiol, carboxyl, sulfate, substituted or unsubstituted amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, alkyl, alkenyl, alkynyl, acyl, acyloxy, acylamino, aryl, heteroaryl, carbocyclyl, heterocyclyl, aralkyl, hetaralkyl, carbocyclylalkyl, and heterocyclylalkyl.

In certain embodiments, D is a substituted or unsubstituted heteroaryl group. In other embodiments, D is a substituted or unsubstituted carbocyclyl group. In additional embodiments, D is a substituted or unsubstituted heterocyclyl group.

In some embodiments, independently for each occurrence, M is substituted or unsubstituted methylene or O, particularly substituted or unsubstituted methylene. In certain embodiments, M is unsubstituted for each occurrence.

In certain embodiments, n is 1 to 3, for example 2.

In certain embodiments, X¹ and X³ are absent. In some embodiments, X² and X⁴ are, independently, NR′ or O, for example X² and X⁴ may both be NR′, such as NH. In certain embodiments, X¹ and X³ are absent and X² and X⁴ are NH.

In some embodiments, Y′ and Y² are C═O.

According to another aspect of the present invention, there is provided a pharmaceutical composition comprising a compound of the invention and a pharmaceutically acceptable carrier or diluent.

In accordance with a further aspect of the present invention, there is provided a method for modulating cell proliferation, preferably inhibiting cell proliferation comprising administering an effective amount of a compound of the invention to a cell or animal in need thereof. The invention also includes a use of a compound of the invention to modulate cell proliferation, preferably inhibit cell proliferation. The invention further includes a use of a compound of the invention to prepare a medicament to modulate cell proliferation, preferably inhibit cell proliferation.

In a preferred embodiment, the present invention provides a method of inhibiting the proliferation of a cancer cell comprising administering an effective amount of a compound of the invention to a cell or animal in need thereof. The cancer cell treated may be any type of cancer including a leukemia, a lymphoma, myeloma, metastatic carcinoma, sarcoma or any other malignant transformation or any other malignancy. The invention also includes a use of a compound of the invention to modulate cancer cell proliferation, preferably inhibit cancer cell proliferation. The invention further includes a use of a compound of the invention to prepare a medicament to modulate cancer cell proliferation, preferably inhibit cancer cell proliferation.

In another aspect, the invention provides a method of modulating tyrosine kinase activity in a cell by administering an effective amount of a compound of the invention. In a further aspect, the invention provides a method of inhibiting tyrosine kinase activity in a cell by administering an effective amount of a compound of the invention. The present invention also provides a use of a compound of the invention to modulate, preferably inhibit, tyrosine kinase activity. The present invention further provides a use of a compound of the invention to prepare a medicament to modulate tyrosine kinase activity, preferably inhibit tyrosine kinase activity. It is appreciated that the inhibition of cell growth by the compounds of the invention may be effected by other mechanisms.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

I. Definitions

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group 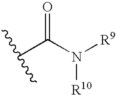
wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

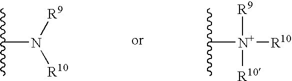

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group 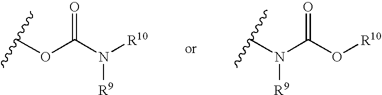
wherein R⁹ and R¹⁰ independently represent hydrogen or a hydrocarbyl group.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—R⁹, wherein R⁹ represents a hydrocarbyl group.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “sulfate” is art-recognized and refers to the group —OSO₃H, or a pharmaceutically acceptable salt thereof.

The term “sulfonamide” is art-recognized and refers to the group represented by the general formulae 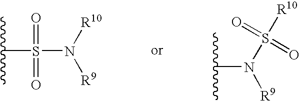
wherein R⁹ and R¹⁰ independently represents hydrogen or hydrocarbyl.

The term “sulfoxide” is art-recognized and refers to the group —S(O)—R⁹, wherein R⁹ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R⁹, wherein R⁹ represents a hydrocarbyl.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula 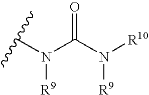
wherein R⁹ and R¹⁰ independently represent hydrogen or a hydrocarbyl.

The term “animal” as used herein includes all members of the animal kingdom including human. The animal is preferably a human.

The term “cancer cells” as used herein includes all forms of cancer or neoplastic disease.

The term “a cell” as used herein includes a plurality of cells. Administering a compound to a cell includes in vivo, ex vivo, and in vitro administration.

The term “compound of the invention” as used herein includes any compound of Formula I as defined herein (including all salts, solvates or hydrates thereof) as well as any compound whose structure is specifically depicted herein (including all salts, solvates or hydrates thereof).

To “inhibit” or “suppress” or “reduce” a function or activity, such as cancer cell proliferation, is to reduce the function or activity when compared to otherwise same conditions except for a condition or parameter of interest, or alternatively, as compared to another conditions.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use. Each carrier must be “acceptable” in the sense of being compatible with other ingredients of the formulation and not injurious to the patient.

Some examples of materials which can serve as pharmaceutically acceptable carriers include (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salt” means an acid addition salt or a basic addition salt which is suitable for or compatible with the treatment of patients.

The term “pharmaceutically acceptable acid addition salt” as used herein means any non-toxic organic or inorganic salt of any base compounds represented by Formula I or II. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I or II are more soluble in water and various hydrophilic organic solvents, and generally demonstrate higher melting points in comparison to their free base forms. The selection of the appropriate salt will be known to one skilled in the art. Other non-pharmaceutically acceptable salts, e.g. oxalates, may be used, for example, in the isolation of compounds of Formula I or II for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or II or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The term “solvate” as used herein means a compound of Formula I or II, or a pharmaceutically acceptable salt of a compound of Formula I or II, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”.

The term a “sufficient amount” or an “effective amount” of an agent as used herein is that amount sufficient to effect beneficial or desired results, including clinical results, and, as such, an “effective amount” depends upon the context in which it is being applied. For example, in the context of administering an agent that inhibits cancer cell proliferation, an effective amount of an agent is, for example, an amount sufficient to achieve such a reduction in cancer cell proliferation as compared to the response obtained without administration of the agent.

A “therapeutically effective amount” of a compound with respect to the subject method of treatment, refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

II. Compounds of the Invention

Compounds which are useful in modulating cell proliferation are disclosed. As such the compounds are useful in treating cell proliferative diseases such as cancer.

Accordingly, the present invention provides methods and compositions employing compounds of Formula I or a salt, solvate, or hydrate thereof:

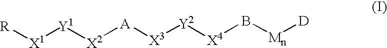

wherein, as valence and stability permit,

R is a substituted or unsubstituted alkyl, alkenyl, or alkynyl group or a substituted or unsubstituted carbocyclyl, heterocyclyl, aryl, or heteroaryl group;

A is a substituted or unsubstituted aryl or heteroaryl group;

B is a substituted or unsubstituted heteroaryl group;

D is a substituted or unsubstituted carbocyclyl, heterocyclyl, aryl, or heteroaryl group;

X¹, X², X³, and X⁴ each independently represent a direct bond, NR′, or O;

Y¹ and Y², independently, each represent C═O, C═S, C═NR′, or SO₂.

M, independently for each occurrence, is a substituted or unsubstituted methylene group, NR′, O, or S, preferably selected so that no two heteroatoms occur in adjacent positions, more preferably with at least two carbon atoms between any nitrogen atom and another heteroatom;

R′, independently for each occurrence, is H or a substituted or unsubstituted lower alkyl group, preferably H or unsubstituted lower alkyl; and

n is an integer from 1 to 6,

or a pharmaceutically acceptable salt thereof.

In certain embodiments, R is a substituted or unsubstituted alkyl group, such as a substituted or unsubstituted lower alkyl group, for example, a substituted or unsubstituted methyl, ethyl, propyl group, for example isopropyl. In some embodiments, R is a substituted alkyl group, for example, an alkyl group substituted by one or more rings, such as carbocyclyl, aryl, heterocyclyl, or heteroaryl group.

In further embodiments, R is a substituted or unsubstituted alkenyl group. For example, R may be an alkenyl group substituted by one or more groups such as carbocyclyl, aryl, heterocyclyl, or heteroaryl.

In other embodiments, R is a substituted or unsubstituted alkynyl group. For example, R may be an alkynyl group substituted by one or more groups such as carbocyclyl, aryl, heterocyclyl, or heteroaryl.

In other embodiments R is a substituted or unsubstituted carbocyclyl group, such as cyclopentyl and cyclohexyl groups.

R may also be a substituted or unsubstituted heterocyclyl group, for example, containing from 1 to 3 heteroatoms selected from N, O, and S. For example, R may be a substituted or unsubstituted piperidine, piperazine, pyrrolidine, morpholine, lactone, lactam, or a saturated furan or pyran group.

In certain embodiments, R is a substituted or unsubstituted aryl group, such as a substituted or unsubstituted phenyl, or naphthyl group.

In other embodiments, R is a substituted or unsubstituted heteroaryl group, such as a substituted or unsubstituted pyrrole, furan, thiophene, imidazole, oxazole, thiazole, pyrazole, pyridine, pyrazine, pyridazine, or pyrimidine group. R may also be a substituted or unsubstituted polycyclic heteroaryl group, such as substituted or unsubstituted benzofuran, benzothiophene, indole, quinoline, quinazoline, quinoxaline, napthyridine, and the like.

In some embodiments, A comprises a substituted or unsubstituted 5- or 6-membered ring, particularly a 6-membered ring. A substituted or unsubstituted 5- or 6 -membered ring of A may be in a monocyclic group, such as in a benzene or pyridine group, or in a polycyclic group, such as in a naphthalene or quinoline group. In certain embodiments, A is a substituted or unsubstituted aryl group, such as a substituted or unsubstituted benzene ring. In alternate embodiments, A is a substituted or unsubstituted heteroaryl group, such as a substituted or unsubstituted pyrrole, furan, thiophene, imidazole, oxazole, thiazole, pyrazole, pyridine, pyrazine, pyridazine, or pyrimidine group. In some of embodiments where A comprises a substituted or unsubstituted 6-membered ring, X² and X³ are disposed in a meta relationship on the 6-membered ring, for example, on a benzene or pyridine ring. In other embodiments, X² and X³ are disposed in a para or ortho relationship on the 6-membered ring.

In certain embodiments A is unsubstituted. In alternate embodiments, A is substituted, for example, with one or more groups selected from halogen, nitro, cyano, hydroxyl, thiol, carboxyl, sulfate, substituted or unsubstituted amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, alkyl, alkenyl, alkynyl, acyl, acyloxy, acylamino, aryl, heteroaryl, carbocyclyl, heterocyclyl, aralkyl, hetaralkyl, carbocyclylalkyl, and heterocyclylalkyl.

In certain embodiments, B comprises a substituted or unsubstituted 5- or 6-membered ring, particularly a 5-membered ring. In some embodiments, B comprises 1-3 heteroatoms selected from O, S, and N. For example, B may be a substituted or unsubstituted thiophene, furan, pyrrole, pyrazole, imidazole, triazole, tetrazole, thiazole, isothiazole, thiadiazole, oxazole, isoxazole, or oxadiazole group, particularly a thiadiazole group. In some instances where B comprises a substituted or unsubstituted 5-membered ring, X⁴ and Mₙ are disposed in a 1,3 relationship on B.

In some embodiments, B is unsubstituted. In alternate embodiments, B is substituted with one or more groups selected from halogen, nitro, cyano, hydroxyl, thiol, carboxyl, sulfate, substituted or unsubstituted amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, alkyl, alkenyl, alkynyl, acyl, acyloxy, acylamino, aryl, heteroaryl, carbocyclyl, heterocyclyl, aralkyl, hetaralkyl, carbocyclylalkyl, and heterocyclylalkyl.

In certain embodiments B represents 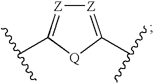
where Z, independently for each occurrence, is N or CR″, particularly N; Q is NR′, O, or S, particularly O or S; and R″, independently for each occurrence, is H or a substituted or unsubstituted lower alkyl group. In some embodiments, Z is N for both occurrences of B.

In certain embodiments, D is a substituted or unsubstituted aryl group, for example, a substituted or unsubstituted phenyl group.

In certain embodiments, D is a substituted or unsubstituted heteroaryl group. For example, D may be a substituted or unsubstituted pyrrole, furan, thiophene, imidazole, oxazole, thiazole, pyrazole, pyridine, pyrazine, pyridazine, or pyrimidine group.

In other embodiments, D is a substituted or unsubstituted carbocyclyl group, such as a substituted or unsubstituted cyclopentane or cyclohexane group.

In additional embodiments, D is a substituted or unsubstituted heterocyclyl group, for example, containing from 1 to 3 heteroatoms selected from N, O, and S. For example, D may be a substituted or unsubstituted piperidine, piperazine, pyrrolidine, morpholine, lactone, lactam, or a saturated furan or pyran group.

In some embodiments, D is unsubstituted. In alternate embodiments, D is substituted with one or more groups selected from halogen, nitro, cyano, hydroxyl, thiol, carboxyl, sulfate, substituted or unsubstituted amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, alkyl, alkenyl, alkynyl, acyl, acyloxy, acylamino, aryl, heteroaryl, carbocyclyl, heterocyclyl, aralkyl, hetaralkyl, carbocyclylalkyl, and heterocyclylalkyl. In some embodiments, D is substituted with polar groups or charged groups know or predicted to improve aqueous solubility. For example, D may be substituted with carboxy, amino (such as primary, secondary and tertiary amino groups, morpholine, piperazine, pyrrolidine, and imidazole groups, etc.), sulfate, and/or phosphate groups. D may also be substituted with groups that are capable of forming acid or base addition salts.

In some embodiments, independently for each occurrence, M is substituted or unsubstituted methylene or O, particularly substituted or unsubstituted methylene. For example, M may be methylene substituted with one or more halogens or ═O groups (i.e. M may be a carbonyl). In certain embodiments, M is unsubstituted for each occurrence.

In certain embodiments, n is 1 to 3, for example 2.

In certain embodiments, X¹ or X³ or both are absent. In some embodiments, X² and X⁴ are, independently, NR′ or O, for example X² or X⁴ or both may be NR′, such as NH. In certain embodiments, X¹ and X³ are absent and X² and X⁴ are NH.

In some embodiments, Y¹ or Y² or both are C═O. In some embodiments, Y² is C═NR′.

In specific embodiments of the present invention, a compound of the invention is selected from:

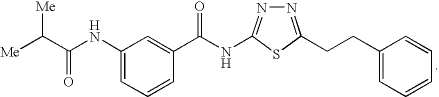

The present invention includes within its scope, prodrugs of the compounds of the invention. In general, such prodrugs will be functional derivatives of a compound of the invention which are readily convertible in vivo into the compound from which it is notionally derived. Conventional procedures for the selection and preparation of suitable prodrugs are described, for example, in “Design of Prodrugs” ed. H. Bundgaard, Elsevier, 1985.

Certain compounds of the present invention may exist in particular geometric or stereoisomeric forms. The present invention contemplates all such compounds, including cis- and trans-isomers, R- and S-enantiomers, diastereomers, (d)-isomers, (1)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers.

The present invention includes radiolabeled forms of compounds of the invention, for example, compounds of the invention labeled by incorporation within the structure ³H or ¹⁴C or a radioactive halogen such as ¹²⁵I.

The compounds of the invention may, for example, be derived from an activated cinnamyl compound and an activated cyano-substituted methylene compound. A person skilled in the art, therefore, may wish to provide a generic name for the compounds of the invention based on the cinnamyl moiety. However, generic nomenclature based on the formed acylonitrile moiety, for example, styryl acrylonitrile, would be more proper.

III. Uses

The present invention includes all uses of the compounds of the invention including their use in therapeutic methods and compositions for modulating cell proliferation, their use in diagnostic assays and their use as research tools.

In one aspect, the present invention provides a method for modulating cell proliferation comprising administering an effective amount of a compound of the invention to a cell or animal in need thereof. Preferably, the invention provides a method of inhibiting cell proliferation comprising administering an effective amount of a compound of the invention to a cell or animal in need thereof. In particular, the method of the invention is useful in inhibiting the proliferation of abnormal but not normal cells. Abnormal cells include any type of cell that is causative of or involved in a disease or condition and wherein it is desirable to modulate or inhibit the proliferation of the abnormal cell to treat the disease or condition. Examples of abnormal cells include malignant or cancerous cells as well as cell that over-proliferate in inflammatory conditions.

It has been determined that some of the compounds of the invention are very effective at killing cancer cells while at the same time they do not kill normal cells. These properties make the compounds of the invention extremely useful as anti-cancer agents. Accordingly, in one embodiment, the present invention provides a method of inhibiting the proliferation of a cancer cell comprising administering an effective amount of a compound of the invention to a cell or animal in need thereof.

The cancer cell that can be treated with a compound of the invention may be any type of cancer including, but not limited to, hematopoietic malignancies, including leukemias, lymphomas, and myelomas as well as other types of cancer including sarcomas, carcinomas, melanomas, adenomas, nervous system cancers and genitourinary cancers. Examples of leukemias include acute lymphoblastic leukemia (ALL), acute myelocytic leukemia (AML), acute myelomonocytic leukemia (AMML), chronic myeloid leukemia (CML), chronic lymphocytic leukemia (CLL) and juvenile myelo-monocytic leukemia (JMML). The types of AML that may be treated with the compounds of the invention include cells that express an aberrant Flt-3 gene, such as a gene that contains an activating internal tandem duplication of variable length (12bp-204bp) in the JM domain (Flt3-ITD) or activating point mutations in the TK2 kinase domain (D835Y, Y842C). The types of ALL that may be treated with the compounds of the invention include cells that express a bcr-abl fusion protein, such as Philadelphia positive ALL cells, as well as Philadelphia negative ALL cells. Examples of lymphomas include B-cell Burkitt's lymphoma, Hodgkin's lymphomas, non-Hodgkin's lymphomas, including the Ki-1 positive anaplastic large cell lymphomas, T cell lymphomas and rare lymphomas such as the histiocytic lymphomas. Examples of myelomas include multiple myelomas.

In a specific embodiment, the present invention provides a method of inhibiting the proliferation of a cancer cell comprising administering an effective amount of a compound selected from:

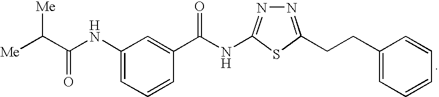

One skilled in the art can determine which compounds of the invention would have therapeutic utility, for example, in inhibiting cell proliferation in any type of cancer or cell proliferative disorder. Accordingly, the methods, uses, and compositions of the invention are meant to include only those compounds having the desired effect.

Another aspect of the invention relates to a method for ex vivo purging, comprising administering a compound of Formula I. For ex vivo administration, bone marrow cells may be removed from a patient with cancer and purged ex vivo with a compound of the invention. Such a purging will kill the tumor cells while leaving the normal bone marrow cells intact. After purging, the cells can be washed and reintroduced into the patient.

During ex vivo purging assays the cells may be exposed to relatively high doses of the compounds (about 50 μM to about 100 μM) for short (about 1 to about 24 hours) periods of time, resulting in the elimination of cancer cell growth, while normal bone marrow cells exposed to the same doses over the same period of time are preferably relatively unaffected. Cancer cell death is effected by the induction of apoptosis. Accordingly, in another aspect of the invention, there is provided a method for killing cancer cells by ex vivo treatment of bone marrow from a patient with cancer with a compound of Formula I, and then re-introducing the treated (or purged) bone marrow into the patient.

In addition to cancer, the compounds of the invention are useful in treating other conditions involving aberrant or abnormal cell proliferation. Other cell proliferative disorders that may be treated by the present invention include inflammatory diseases, allergies, autoimmune disease, graft rejection psoriasis, restenosis, artherosclerosis, and any other disorder wherein it is desirable to inhibit, prevent, or suppress cell growth. Compounds of the invention may be tested for their efficacy in a particular cell proliferation disorder using assays and techniques known to those of skill in the art. For example, the following references provide assays for various conditions: Rheumatoid Arthritis: C. S. Kasyapa et al., J. Immunology (1999) 163, 8236; Allergy: T. Adachi et al., J. Immunology (1999) 163, 939; Psoriasis: R. Üchert, J. Immunology (2000) 165, 224. Psoriasis: A. H. Enk., Int. Arch. Allergy Immunol. (2000) 123, 275.

Another aspect of the invention relates to a method for treating chronic hematological proliferative diseases such as leukemia or polycythemia vera. In certain embodiments, the chronic hematological proliferative disease is Jak-2 dependent. In certain such embodiments, the Jak-2 dependent chronic hematological proliferative disease is selected from atypical chronic myeloid leukemia, chronic myelomonocytic leukemia, chronic eosinophilic leukemia, acute myeloid leukemia, acute lymphoblastic leukemia, myelodysplasia, and hyper-eosinophilic syndrome.

The compounds of the invention are tyrosine kinase modulators and are useful in modulating tyrosine kinase activity, including the inhibition of tyrosine kinase activity, for the treatment of various conditions such as all proliferative disorders as mentioned above. Accordingly, the invention provides a method of modulating tyrosine kinase activity by administering an effective amount of a compound of the invention to a cell or animal in need thereof. In a further aspect, the invention provides a method of inhibiting tyrosine kinase activity by administering an effective amount of a compound of the invention to a cell or animal in need thereof.

While the compounds of the invention may act by inhibiting tyrosine kinase activity, one of skill in the art will appreciate that other modes or mechanisms of action for the compounds of the invention are possible.

In one aspect, the invention relates to a method of promoting myelopoiesis comprising administering an effective amount of one or more of these myelopoiesis-promoting compounds to hematopoietic cell or an animal in need thereof.

In some embodiments, the hematopoietic cell is hematopoeitic stem cell and the animal is a human patient. In specific embodiments, the compounds are administered to a human patient suffering from, or at risk of primary or secondary neutropenia, including chemotherapy or drug induced neutropenia, neutropenia secondary to malignancy, including G-CSF responsive malignancies. In other embodiments, the compound(s) is administered to a human patient at risk of, or suffering from aplastic anemia, such as Fanconi anemia, or aplasia. In other embodiments, the animal is a human donor of bone marrow cells or peripheral blood stem cells. In other embodiments, one or more of these myelopoiesis-promoting compounds is administered to a human patient in need of bone marrow cell or peripheral blood stem cell transplant before or after the transplant.

In another aspect, the invention provides a method of promoting myelopoiesis ex vivo comprising administering an effective amount of one or more of these myelopoiesis-promoting compounds to a hematopoietic cell. In one embodiment, the cell is hematopoietic stem cell. In specific embodiments, the hematopoietic cell is from the bone marrow or peripheral blood stem cells of a donor, or the bone marrow or peripheral blood stem cells of a patient in need of autologous bone marrow or peripheral blood stem cell transplant.

In another aspect, the invention provides a method of treating a patient suffering from or at risk of neutropenia, aplastic anemia, or aplasia, comprising administering an effective amount of one or more of these myelopoiesis-promoting compounds to said patient. In another aspect, the invention provides a method of treating a patient suffering from or at risk of neutropenia, aplastic anemia, or aplasia, comprising introducing hematopoietic cells to the patient wherein one or more of these myelopoiesis-promoting compounds has been administered to the cells ex vivo in an amount effective to promote myelopoiesis. The hematopoietic cells may be from the bone marrow or peripheral blood stem cells of a donor or of the patient.

In another aspect, the invention relates to use of one or more of these myelopoiesis-promoting compounds to promote myelopoiesis. The invention also relates to use of one or more of these myelopoiesis-promoting compounds for preparing a medicament to promote myelopoiesis. Yet in another aspect, the invention relates to use of one or more of these myelopoiesis-promoting compounds to treat neutropenia, aplastic anemia, or aplasia, and the use of one or more of these myelopoiesis-promoting compounds to prepare a medicament to treat neutropenia, aplastic anemia, or aplasia.

In another aspect, the invention provides a kit comprising one or more of these myelopoiesis-promoting compounds and instructions for use, including to promote myelopoiesis and to treat neutropenia, aplastic anemia, or aplasia.

The compounds of the invention are preferably formulated into pharmaceutical compositions for administration to human subjects in a biologically compatible form suitable for administration in vivo. Accordingly, in another aspect, the present invention provides a pharmaceutical composition comprising a compound of the invention in admixture with a suitable diluent or carrier.

The compositions containing the compounds of the invention can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffered solutions with a suitable pH and iso-osmotic with the physiological fluids.

The compounds of this invention may be used in the form of the free base, in the form of salts, solvates and as hydrates. All forms are within the scope of the invention. Acid addition salts may be formed and provide a more convenient form for use; in practice, use of the salt form inherently amounts to use of the base form. The acids which can be used to prepare the acid addition salts include preferably those which produce, when combined with the free base, pharmaceutically acceptable salts, that is, salts whose anions are non-toxic to the animal organism in pharmaceutical doses of the salts, so that the beneficial properties inherent in the free base are not vitiated by side effects ascribable to the anions. Although pharmaceutically acceptable salts of the basic compounds are preferred, all acid addition salts are useful as sources of the free base form even if the particular salt per se is desired only as an intermediate product as, for example, when the salt is formed only for the purposes of purification and identification, or when it is used as an intermediate in preparing a pharmaceutically acceptable salt by ion exchange procedures.

Pharmaceutically acceptable salts within the scope of the invention include those derived from the following acids; mineral acids such as hydrochloric acid, sulfuric acid, phosphoric acid and sulfamic acid; and organic acids such as acetic acid, citric acid, lactic acid, tartaric acid, malonic acid, methanesulfonic acid, ethanesulfonic acid, benzenesulfonic acid, p-toluenesulfonic acid, cyclohexylsulfamic acid, quinic acid, and the like.

In accordance with the methods of the invention, the described compounds or salts or solvates thereof may be administered to a patient in a variety of forms depending on the selected route of administration, as will be understood by those skilled in the art. The compositions of the invention may be administered orally or parenterally. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

A compound of the invention or a salt or solvate thereof may be orally administered, for example, with an inert diluent or with an assimilable edible carder, or it may be enclosed in hard or soft shell gelatin capsules, or it may be compressed into tablets, or it may be incorporated directly with the food of the diet. For oral therapeutic administration, the compound of the invention may be incorporated with excipient and used in the form of ingestible tablets, buccal tablets, troches, capsules, elixirs, suspensions, syrups, wafers, and the like.

A compound of the invention may also be administered parenterally or intraperitoneally. Solutions of a compound of the invention as a free base or pharmacologically acceptable salt or solvate can be prepared in water suitably mixed with a surfactant such as hydroxypropylcellulose. Dispersions can also be prepared in glycerol, liquid polyethylene glycols, DMSO, and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (1990-18th edition) and in The United States Pharmacopeia: The National Formulary (USP 24 NF19) published in 1999.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

The compounds of the invention may be administered to an animal alone or in combination with pharmaceutically acceptable carriers, as noted above, the proportion of which is determined by the solubility and chemical nature of the compound, chosen route of administration and standard pharmaceutical practice.

The dosage of the compounds and/or compositions of the invention can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the animal to be treated. One of skill in the art can determine the appropriate dosage based on the above factors. The compounds of the invention may be administered initially in a suitable dosage that may be adjusted as required, depending on the clinical response. As an example, the compounds of the invention can be administered in a range from about 1 nanomolar to about 100 micromolar, preferably 50 nanomolar to 50 micromolar. For ex vivo treatment of cells over a short period, for example for 30 minutes to 1 hour or longer, higher doses of compound may be used than for long term in vivo therapy; for example, concentrations of 50 μM or higher may be used.

The present invention also includes a use of a compound or composition of the invention in order to inhibit cell proliferation, preferably cancer cell proliferation. The present invention further includes a use of a compound or a composition of the invention to prepare a medicament to inhibit cell proliferation, preferably cancer cell proliferation.

The compounds of the invention can be used alone or in combination with other agents that modulate tyrosine kinase activity or in combination with other types of treatment (which may or may not modulate tyrosine kinase activity) for cell proliferative disorders. Agents known in the art that inhibit tyrosine kinase activity include, but are not limited to, antisense nucleic acid and ribozymes targeted to nucleic acid encoding a receptor tyrosine kinase, antibodies able to modulate tyrosine kinase activity and other small molecule tyrosine kinase inhibitors such as those described in U.S. Pat. No. 5,891,917, U.S. Pat. No. 5,217,999, U.S. Pat. No. 5,773,476, U.S. Pat. No. 5,935,993, U.S. Pat. No. 5,656,655, U.S. Pat. No. 5,677,329, and U.S. Pat. No. 5,789,427. There are various examples of other types of treatment for cell proliferative disorders currently used to treat different types of cancers. The general treatments are based on the cancer type and do not specifically target tyrosine kinase activity. In a particular aspect of the present invention, the compounds of the invention may be used in combination with other therapies and therapeutics to treat leukemia.

In addition to the above-mentioned therapeutic uses, the compounds of the invention are also useful in diagnostic assays, screening assays and as research tools.

In diagnostic assays the compounds of the invention may be useful in identifying or detecting a cell proliferative disorder. In such an embodiment, the compounds of the invention may be radiolabelled (as hereinbefore described) and contacted with a population of cells. The presence of the radiolabel on the cells may indicate a cell proliferative disorder. In a specific embodiment, the radiolabelled compounds of the invention may be used to detect the presence of cells expressing a bcr-abl fusion protein.

In screening assays, the compounds of the invention may be used to identify other compounds that modulate cell proliferation or tyrosine kinase activity. As research tools, the compounds of the invention may be used in receptor binding assays and assays to study the localization of tyrosine kinases. In such assays, the compounds may also be radiolabelled.

The following non-limiting examples are illustrative of the present invention:

Exemplification

Compounds of the present invention were assayed for activity using a WST-1 cell proliferation assay and/or an Alamar Blue-based assay.

WST-1 Assay

The WST-1 cell proliferation assay employed was obtained from Chemicon International, Inc. of Temecula, Calif. Further information on assay materials and procedure is provided at www.chemicon.com. The WST-1 cell proliferation assay relies on the formation of a formazan from a tetrazolium salt by cellular enzymes, whereby the amount of the obtained formazan dye as an indicator of viability is directly proportional to the number of metabolically active cells in culture.

Following 72 hours incubation of the respective cells in 50 μL/well with test compound, 10 μL of WST-1 reagent was added to each well and its contents briefly mixed. The cells were then incubated for 2 hours before absorbance of the dye was measured at 450 nm using a commercial UV/VIS microplate reader (Molecular Devices).

Alamar Blue Assay

The Alamar Blue assay employed the CellTiter-Blue™ Cell Viability Assay from Promega Corporation of Madison, Wis. Further information on assay materials and procedure is provided at www.promega.com. The Alamar Blue-based CellTiter-Blue assay indicates cell viability by measuring the ability of cells in culture to reduce resazurin to resorufin, whereby the intensity of the fluorescence signal is directly proportional to the number of live cells.

Tumor cells were seeded at the recommended seeding concentrations in 50 μL/well. Test article solutions were added 24 hours after seeding and the cells were incubated for 72 hours with the test compound. Following addition of CellTiter-Blue Reagent (20 μL/well) and brief mixing, cells were incubated for another 4 hours before fluorescence was measured (Ex/Em of 560/590 nm) using a commercial UV/VIS microplate reader (Molecular Devices).

Data for Compound A

(A)

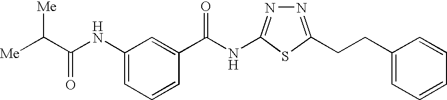

| | | |
|---|---|---|
| | | IC50 (μM) for Cell |
| Cell Line | Cell Type | Proliferation |

Activity of Compound A in Human Solid Tumor Cell Lines

| | | |
|---|---|---|
| ACHN | Renal cell adenocarcinoma | 5.00 |
| 786-0 | Renal cell adenocarcinoma | 4.34 |
| OVCAR-5 | Ovarian carcinoma | 6.65 |
| SKOV-3 | Ovarian adenocarcinoma | 3.50 |
| DU145 | Prostate carcinoma | 6.14 |
| PC3 | Prostate adenocarcinoma | 2.35 |
| A549 | Lung carcinoma | 19.06 |
| NCI-H460 | Large cell lung carcinoma | 2.32 |
| HCT-116 | Colorectal carcinoma | 17.32 |
| MDA-MB453 | Metastatic breast carcinoma | 1.71 |
| MCF-7 | Epithelial mammary | >20.00 |
| | adenocarcinoma | |
| MiaPACA-2 | Pancreas carcinoma | 3.36 |
| PANC-1 | Pancreas duct epithelial | >20.00 |
| | carcinoma | |
| SK-MEL-5 | Malignant melanoma | 2.39 |
| DAOY | Cerebellar medulloblastoma | 2.70 |

Activity of Compound A in Human Acute Leukemia Cell Lines

| | | |
|---|---|---|
| OCI-AML2 | hAML | 0.35 |
| MOLM-13 | hAML | 0.83 |
| KASUMI-1 | hAML | 20.00 |
| SKM-1 | hAML | 0.60 |
| MV4-11 | hAMoL | 0.93 |
| CCRF-CEM | hT-ALL | 0.97 |
| MOLT-4 | hT-ALL | 0.34 |
| PEER | hT-ALL | >20.00 |
| C1 | hB-ALL | 0.57 |
| SUP-B15 | hB-ALL | 4.82 |
| RS4-11 | hB-ALL | 0.13 |

hAML human acute myeloid leukemia
hAMoL human acute myelo-monocytic leukemia
hT-ALL human T-cell derived lymphoblastic leukemia
hB-ALL human B-cell derived acute lymphoblastic leukemia

All publications and patents cited herein are hereby incorporated by reference in their entirety.

Those skilled in the art will recognize, or be able to ascertain using no more than routine experimentation, many equivalents to the specific embodiments of the invention described herein. Such equivalents are intended to be encompassed by the following claims.

We claim:

1. A method for inhibiting cell proliferation or treating cancer, comprising administering a compound according to Formula I: 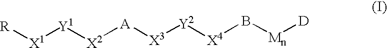
wherein, as valence and stability permit,
R is a substituted or unsubstituted alkyl, alkenyl, or alkynyl group or a substituted or unsubstituted carbocyclyl, heterocyclyl, aryl, or heteroaryl group
A is a substituted or unsubstituted aryl or heteroaryl group;
B is a substituted or unsubstituted heteroaryl group;
D is a substituted or unsubstituted carbocyclyl, heterocyclyl, aryl, or heteroaryl group;
X¹, X², X³, and X⁴ each independently represent a direct bond, NR′, or O;
Y¹ and Y², independently, each represent C═O, C═S, C═NR′, or SO₂.
M, independently for each occurrence, is a substituted or unsubstituted methylene group, NR′, O, or S;
R′, independently for each occurrence, is H or a substituted or unsubstituted lower alkyl group; and
n is an integer from 1 to 6,
or a pharmaceutically acceptable salt thereof.

2. The method of claim 1, wherein R is a substituted or unsubstituted alkyl group.

3. The method of claim 2, wherein R is a substituted or unsubstituted lower alkyl group.

4. The method of claim 3, wherein R is an isopropyl group.

5. The method of claim 1, wherein A comprises a substituted or unsubstituted 6-membered ring.

6. The method of claim 1, wherein A is a substituted or unsubstituted aryl group.

7. The method of claim 6, wherein A is a substituted or unsubstituted benzene ring.

8. The method of claim 7, wherein X² and X³ are disposed in a meta relationship on the benzene ring.

9. The method of claim 7, wherein A is unsubstituted.

10. The method of claim 7, wherein A is substituted with one or more groups selected from halogen, nitro, cyano, hydroxyl, thiol, carboxyl, sulfate, substituted or unsubstituted amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, alkyl, alkenyl, alkynyl, acyl, acyloxy, acylamino, aryl, heteroaryl, carbocyclyl, heterocyclyl, aralkyl, hetaralkyl, carbocyclylalkyl, and heterocyclylalkyl.

11. The method of claim 1, wherein B comprises a substituted or unsubstituted 5-membered ring.

12. The method of claim 11, wherein B comprises 1-3 heteroatoms selected from O, S, and N.

13. The method of claim 12, wherein B is a substituted or unsubstituted thiophene, furan, pyrrole, pyrazole, imidazole, triazole, tetrazole, thiazole, isothiazole, thiadiazole, oxazole, isoxazole, or oxadiazole group.

14. The method of claim 13, wherein B is a thiadiazole group.

15. The method of claim 11, wherein X⁴ and Mₙ are disposed in a 1,3 relationship on B.

16. The method of claim 11, wherein B is unsubstituted.

17. The method of claim 11, wherein B is substituted with one or more groups selected from halogen, nitro, cyano, hydroxyl, thiol, carboxyl, sulfate, substituted or unsubstituted amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, alkyl, alkenyl, alkynyl, acyl, acyloxy, acylamino, aryl, heteroaryl, carbocyclyl, heterocyclyl, aralkyl, hetaralkyl, carbocyclylalkyl, and heterocyclylalkyl.

18. The method of claim 13, wherein
B represents 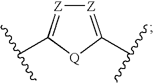
Z, independently for each occurrence, is N or CR″;
Q is NR′, O, or S; and
R″, independently for each occurrence, is H or a substituted or unsubstituted lower alkyl group.

19. The method of claim 18, wherein Z is N for both occurrences.

20. The method of claim 18, wherein Q is O or S.

21. The method of claim 1, wherein D is a substituted or unsubstituted aryl group.

22. The method of claim 21, wherein D is a substituted or unsubstituted phenyl group.

23. The method of claim 22, wherein D is unsubstituted.

24. The method of claim 22, wherein D is substituted with one or more groups selected from halogen, nitro, cyano, hydroxyl, thiol, carboxyl, sulfate, substituted or unsubstituted amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, alkyl, alkenyl, alkynyl, acyl, acyloxy, acylamino, aryl, heteroaryl, carbocyclyl, heterocyclyl, aralkyl, hetaralkyl, carbocyclylalkyl, and heterocyclylalkyl.

25. The method of claim 1, wherein D is a substituted or unsubstituted heteroaryl group.

26. The method of claim 1, wherein D is a substituted or unsubstituted carbocyclyl group.

27. The method of claim 1, wherein D is a substituted or unsubstituted heterocyclyl group

28. The method of claim 1, wherein M is methylene for each occurrence.

29. The method of claim 28, wherein M is unsubstituted for each occurrence.

30. The method of claim 1, wherein n is 1 to 3.

31. The method of claim 30, wherein n is 2.

32. The method of claim 1, wherein X¹ and X³ are absent.

33. The method of claim 32, wherein X² and X⁴ are, independently, NR′ or O.

34. The method of claim 33, wherein X² and X⁴ are NR′.

35. The method of claim 1, wherein Y¹ and Y² are C═O.

36. The method of claim 1 for inhibiting cell proliferation.

37. The method of claim 36, wherein the cells are cancer cells.

38. The method of claim 1 for treating cancer.

39. The method of claim 37 or 38, wherein said cancer is a hematopoietic cell cancer.

40. The method of claim 37 or 38, wherein said cancer is a leukemia, a lymphoma, a myeloma or a carcinoma.

41. The method of claim 40, wherein said leukemia is acute lymphoblastic leukemia, Philadelphia+ leukemia, Philadelphia− leukemia, acute myelocytic leukemia, chronic myeloid leukemia, chronic lymphocytic leukemia or juvenile myelomonocyte leukemia.

42. The method of claim 40, wherein said leukemia is acute lymphoblastic leukemia.
